(19) United States
(12) Patent Application Publication
(10) Pub. No.: US 2016/0178649 A1
(43) Pub. Date: Jun. 23, 2016

(54) METHOD AND SYSTEM FOR SENSING AND DETECTING A TARGET MOLECULE

(71) Applicants: Ranganathan Shashidhar, Needham Heights, MA (US); Yufeng Ma, Needham Heights, MA (US); James A. Kane, Needham Heights, MA (US)

(72) Inventors: Ranganathan Shashidhar, Needham Heights, MA (US); Yufeng Ma, Needham Heights, MA (US); James A. Kane, Needham Heights, MA (US)

(21) Appl. No.: 14/885,425

(22) Filed: Oct. 16, 2015

Related U.S. Application Data
(60) Provisional application No. 62/064,681, filed on Oct. 16, 2014.

(51) Int. Cl. G01N 33/74; B01L 3/00; G01N 27/02

(52) U.S. Cl. CPC G01N 33/74; G01N 27/021; B01L 3/502715; B01L 3/502761; G01N 2333/575; G01N 2600/00; B01L 2300/16; B01L 2300/12; B01L 2300/0627

(57) ABSTRACT

Biosensors and sensing methods that overcome the disadvantages, poor chemical, physical and long-term stability, hatch to batch variability and high cost sensor of these teachings for detecting and recognizing target molecules includes a capture and release component and a sensing component. The capture release component includes a structure having molecularly imprinted polymer nanoparticles disposed on the structure, the structure being configured to receive a target fluid having the target molecules, the target molecules being captured by one of a molecularly imprinted polymer or molecularly imprinted polymer nanoparticles, and a source of a release solvent configured to release the target molecules captured by the molecularly imprinted polymer nanoparticles, the release solvent and released target molecules constituting a release solution. The sensing component includes a sensor surface having a layer of molecular imprinted polymer disposed on the sensor surface; and a sensing circuit.

### CROSS REFERENCE TO RELATED APPLICATIONS

This application claims priority of U.S. Provisional Application No. 62/064,681, filed Oct. 16, 2014, entitled METHOD AND SYSTEM FOR SENSING AND DETECTING A TARGET MOLECULE, which is incorporated by reference herein in its entirety for all purposes.

### STATEMENT REGARDING FEDERALLY SPONSORED RESEARCH OR DEVELOPMENT

This invention was made partially with U.S. Government support from the U.S. Army Contracting Command Redstone under Contract No. W31P4Q-14-C-0004, The federal government may have certain rights in the invention.

### BACKGROUND

These teachings relate generally to methods and systems for detecting a target molecule in the target fluid.

Molecular recognition is fundamental to a number of biological mechanisms. Sensors for molecular recognition are referred to herein as biosensors, although that name should not be considered limiting.

A biosensor typically has two components, a recognizing element that interacts with the target molecule and a transducing element that converts the interaction into a quantifiable effect. Some common recognizing elements are based on antibody, enzymatic, cellular or bio receptor interactions. Typical transducing elements are electrochemical optical and dielectric elements.

Although a number of biosensors configured as described above have been used, there are some basic disadvantages-poor chemical, physical and long-term stability, batch to batch variability and high cost. There is a need for biosensors that will overcome these disadvantages.

In order to provide a concrete example, the detection of oxytocin levels is described herein below.

Oxytocin is a peptide hormone widely known for its role in reproduction and child birth. In fact, the word “oxytocin” was coined from the Greek words meaning “quick birth” after its uterine-contracting properties were discovered by Dale. More recently, the role of oxytocin as a neuromodulator in the central nervous system of humans has been recognized. It is now known that oxytocin indeed plays a very important role in a variety of complex social behavior. For instance, high peripheral oxytocin levels have been associated with better relationship quality. Oxytocin may also be capable of modulating inflammation and promoting wound repair. It is also realized that the levels of oxytocin can affect human stress behaviors, interpersonal relations, and even wound healing.

While the importance of oxytocin has stimulated a major interest in monitoring oxytocin levels to better understand its role in human and animal behavior, there are some technical issues with regards to the current state-of-the art capability for measuring oxytocin levels.

Specificity Issue: Recent studies have shown that the regulation of oxytocin is a complex process involving two forms of oxytocin. Initially a 12-amino acid hormone is produced. Subsequently, it may be cleaved to a 9-amino acid hormone. This shortened form is the active neuropeptide credited with oxytocin's behavior-altering effects. While the biological role, if any, of the 12-amino acid pre-hormone is unknown, it has been associated with atypical social behaviors in autism and possibly related to obesity. Hence the measurement method of oxytocin level must have the specificity to distinguish between the 9- and 12-aminoacid forms of oxytocin. Current immunoassays fail to differentiate the neuroactive form from the pre-hormone version. In immunoassays, the specific recognition ability of antibodies relies on a short variable sequence of amino acids at the tips of the Y-structure, which is called the paratope and specific for one particular moiety of the analyte. In the scenario of oxytocin detection, 9- and 12- amino acid forms of oxytocin both bind to the paratope of antibody with a similar affinity because both the 9- and the 12-amino acid versions consist of an identical amino acid tip segment. Consequently, immunoassay cannot discriminate between the 9- and 12-amino acid forms.

Sensitivity issue: Basal blood levels of oxytocin are in the pg/mL range. This low biological level makes accurate measurements of oxytocin difficult. For instance, under normal physiological conditions, oxytocin levels in blood are ˜5 pg/ml and the corresponding salivary concentrations would be 0.25-0.50 pg/ml, which is undetectable by current immunoassay technologies.

Hence, the current immunoassay-based methods do not have either the specificity or sensitivity needed. Oxytocin assays with improved sensitivity and specificity would be the necessary tools to understand the function of this important neurohormone.

### BRIEF SUMMARY

Biosensors and sensing methods that overcome the disadvantages—poor chemical, physical and long-term stability, batch to batch variability and high cost, are disclosed herein below. Oxytocin assays and oxytocin sensing methods with improved sensitivity and specificity are also disclosed herein below.

In one or more embodiments, the sensor of these teachings for detecting and recognizing target molecules includes a capture and release component and a sensing component. The capture release component includes a structure having one of molecularly imprinted polymer layer or molecularly imprinted polymer nanoparticles disposed on the structure, the structure being configured to receive a target fluid having the target molecules, the target molecules being captured by the molecularly imprinted polymer nanoparticles, and a source of a release solvent configured to release the target molecules captured by the molecularly imprinted polymer nanoparticles, the release solvent and released target molecules constituting a release solution. The sensing component includes a sensor surface having a layer of molecular imprinted polymer disposed on the sensor surface; the layer of molecularly imprinted polymer disposed to receive the release solution, the target molecules binding to the molecularly imprinted polymer, and a sensing circuit configured to detect impedance changes in the layer of molecularly imprinted polymer caused by the binding of the target molecules to the molecularly imprinted polymer.

In one or more embodiments, the method of these teachings includes disposing molecularly imprinted polymer nanoparticles on a surface of a structure, receiving, at the surface, a target fluid having the target molecules, capturing the target molecules in the molecularly imprinted polymer nanoparticles, releasing, after capture, the target molecules from the molecularly imprinted polymer nanoparticles, the target molecules being released into a release solution, providing the release solution to a sensor surface having a layer of molecular imprinted polymer disposed on the sensor surface, the target molecules binding to the layer of molecularly imprinted polymer, and detecting impedance changes in the layer of molecularly imprinted polymer caused by the binding of the target molecules to the molecularly imprinted polymer, the target molecules being detected by the impedance changes.

A number of other embodiments are also disclosed.

For a better understanding of the present teachings, together with other and further objects thereof, reference is made to the accompanying drawings and detailed description and its scope will be pointed out in the appended claims.

### BRIEF DESCRIPTION OF THE DRAWINGS

FIG. 1a shows a graphical schematic representation of one embodiment of the system of these teachings;

FIG. 1b is a graphical schematic representation of a target molecule imprinting as used in these teachings;

FIGS. 2a, 2b show a) a schematic representation of the Randles equivalent circuit as used in the system of these teachings, and b) shows an impedance plot for the Randles equivalent circuit as used in the system of these teachings;

FIG. 3 shows Potential wave form for differential pulse voltammetry (DPV) as of pain from a sensing component in an embodiment of the system of these teachings;

FIG. 4 shows Molecularly-imprinted polymer nanoparticles via emulsion polymerization as used in the system of these teachings;

FIG. 4a shows schematic representation of the protocol designed for MIP coating on the microcolumn array for first-stage purification microfluidics;

FIG. 4b shows a selection of monomers and cross-linkers that can be used in development of a component in the system of these teachings;

FIGS. 4c-4e show schematic representations of a) Template for microcolumn array fabrication, b) microcolumn array and (c) manifold assembly;

FIGS. 5a, 5b show UV-Visible spectra of as-prepared oxytocin solution (solid line) and after capture (dashed line) for 9-(a) and 12-amino acid version (b) respectively;

FIGS. 6a-6e show UV-Visible spectra (a-e) of as-prepared OXT-9 solutions (solid line) and released OXT-9 (dashed line) using releasing solutions with different pH values. Plot of releasing efficiency with pH values is shown in 6f;

FIG. 7a-7e show UV-Visible spectra (a-e) of as-prepared OXT-12 solutions (solid line) and released OXT-12 (dashed line) using releasing solutions with different pH values. Plot of releasing efficiencies with pH values is shown in 7f;

FIG. 8a-8e show a) Schematic of procedures for demonstrating specificity. One of the columns is imprinted with OXT-9 while the other is imprinted with OXT-12. b) UV-Visible spectra: As-prepared OXT-9 (solid), through OXT-12-imprinted column (dashed), and through OXT-9-imprinted column (dotted). c) UV-Visible spectra: As-prepared OXT-12 (solid), through OXT-9-imprinted column (dashed), and through OXT-12-imprinted column (dotted);

FIGS. 9a-9c show Flight high-resolution mass spectra (UPLC-QtoF HRMS) of (a) as-prepared sample solution containing both OXT-9 and OXT-12 forms; (h) sample solution through OXT-12-imprinted column and (c) sample solution through OXT-9-imprinted column. Peaks and its relevant area indicate the amount of OXT-9 or OXT-12 version in solution accordingly;

FIG. 10 is a protocol designed to fabricate detectors of these teachings;

FIGS. 11a-11d show DPV current responses to different concentrations of (a) OXT-9 and (c) OXT-12 in their corresponding optimal releasing solutions, in which pH of 5.3 is for OXT-9 and pH of 8.9 is for OXT-12. Plots of peak current versus oxytocin concentration are shown in (b) and (d) for OXT-9 and OXT-12, respectively;

FIG. 12a-12d show DPV current responses to different concentrations of (a) OXT-9 and (c) OXT-12 version in the corresponding PBS solutions, where pH of 5.3 is for OXT-9 and pH of 8.9 is for OXT-12. Plot of relative peak current (%) versus oxytocin concentration (b) and (d) for 9- and 12-amino acid version, respectively. The relative current change is defined as (I_{c}-Iₒ)/Iₒ*100%, where I_{c} and Iₒ represent the peak current value at corresponding oxytocin level and the peak without oxytocin, respectively; and

FIGS. 13a,13b represent (a) Schematic showing the individual components, (b) drawing of device of these teachings with integrated Functions.

### DETAILED DESCRIPTION

The following detailed description presents the currently contemplated modes of carrying out the invention. The description is not to be taken in a limiting sense, but is made merely for the purpose of illustrating the general principles of the invention, since the scope of the invention is best defined by the appended claims.

As used herein, the singular forms “a,” “an,” and “the” include the plural reference unless the context clearly dictates otherwise.

Except where otherwise indicated, all numbers expressing quantities of ingredients, reaction conditions, and so forth used in the specification and the claims are to be understood as being modified in all instances by the term “about.” Further, any quantity modified by the term “about” or the like should be understood as encompassing a range of ±10% of that quantity unless otherwise specified.

Biosensors and sensing methods that overcome the disadvantages, poor chemical, physical and long-term stability, batch to batch variability and high cost, are disclosed herein below.

In one or more embodiments, the sensor of these teachings for detecting and recognizing target molecules includes a capture and release component and a sensing component. The capture release component includes a structure having ne of molecularly imprinted polymer layer or molecularly imprinted polymer nanoparticles disposed on the structure, the structure being configured to receive a target fluid having the target molecules, the target molecules being captured by the molecularly imprinted polymer nanoparticles, and a source of a release solvent configured to release the target molecules captured by the molecularly imprinted polymer nanoparticles, the release solvent and released target molecules constituting a release solution. The sensing component includes a sensor surface having a layer of molecular imprinted polymer disposed on the sensor surface; the layer of molecularly imprinted polymer disposed to receive the release solution; the target molecules binding to the molecularly imprinted polymer and a sensing circuit configured to detect impedance changes in the layer of molecularly imprinted polymer caused by the binding of the target molecules to the molecularly imprinted polymer. The capture and release components are operatively connected to receive from a fluid source, the fluid being the target fluid or the release solvent. The sensing component is operatively connected to the capture and release components in order to receive the release solution.

In one embodiment, the sensor of these teachings is designed to work in a two-stage scenario. The first stage is “Capture” in which the target molecule is captured from sample solution by a molecularly-imprinted polymer (MIP). A release solution is then introduced to induce changes in charge and conformation of the captured oxytocin to facilitate its release. The release solution subsequently delivers the released target to the “Detection” stage, which consists of another version of molecularly-imprinted polymer.

A principle of the above embodiment is based on the use of different forms of “molecular imprinting technology”. Molecular imprinting, which enables creation of stable and selective “artificial receptors,” is a method for preparing polymers of predetermined selectivity for the separation and analysis of a vast variety of biologically active molecules. This method has also been the focus of attention for peptide and protein extraction and purification. The technique involves the formation of complexes between a print molecule (template) and a functional monomer based on relatively weak, non-covalent interactions. These complexes appear spontaneously in the liquid phase and are then fixed sterically by polymerization with a high degree of cross-linking. After extracting the print molecules from the synthesized polymer, empty recognition sites remain in the polymer matrix and these sites can recognize the original template molecules during subsequent exposure. Molecularly-imprinted materials have been called “antibody mimics” because these systems attempt to mimic the interactions of their natural counterparts and have achieved affinity and selectivity that approach those of natural recognitions.

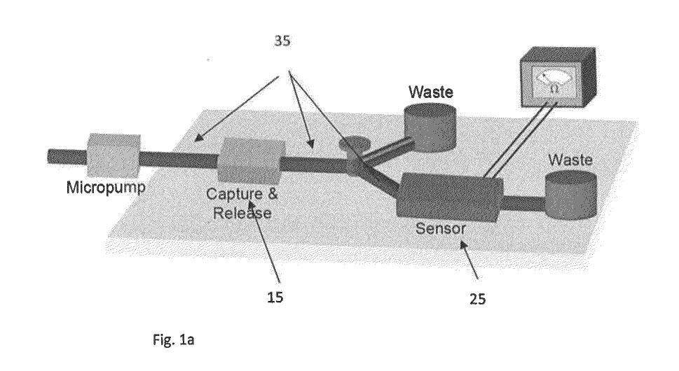

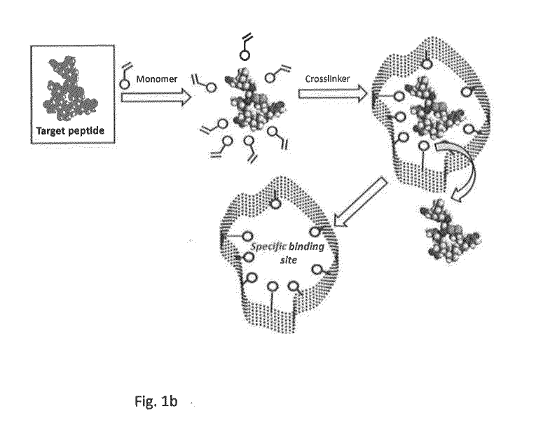

An embodiment of the sensor of these teachings that works in two stages as illustrated in FIG. 1a. The first stage is “Capture,” using the capture and release component 15, in which the target molecule is captured from sample solution by a molecularly-imprinted polymer (MIP). A release solution is then introduced to release the captured molecule (such as a peptide) and deliver the released target to the “Detection” stage 25, which consists of another version of molecularly-imprinted polymer. In this approach, the capturing and detection steps are carried out using two different forms of molecularly-imprinted polymers in which the imprinting is done using two distinctive conformations of the target molecule. The capture and release component is operatively connected by means of a conduit 35 that carries fluid from a source, a micro-pump. The capture release component 15 and the sensor 25 are operatively connected by another conduit 35 that carries fluid, the released target, from the capture release component 15 to the sensor 25.

The circumstance that the conformations and charges of the target molecule (such as a peptide) can be tuned by experimental conditions such as pH and ion concentration forms a principle of the present teachings. Researchers have taken advantage of this property to control nanocrystal growth by tuning peptide conformation (Banerjee, I. A. et al, “Cu nanocrystal growth on peptide nanotubes by biomineralization: Size control of Cu nanocrystals by tuning peptide conformation,” PNAS 2003; 100: 14678-14682, which is incorporated by reference herein in its entirety and for all purposes). By tailoring the properties of polymers (by varying charge distribution or hydrophobicity or hydrophilicity or pore size), the formed specific binding site in molecularly imprinted polymer (MIP) matrix can record the conformation and charge state of the target peptide (see FIG. 1b). Equally importantly, the molecularly imprinted polymer in the purification stage is specifically tailored for the conformation and charge state of the target peptide in physiological conditions while the polymer for the detection is designed to be specific to the target molecule in the releasing solution.

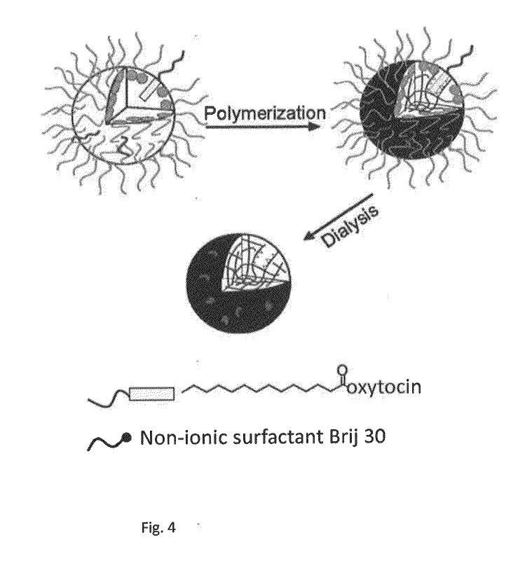

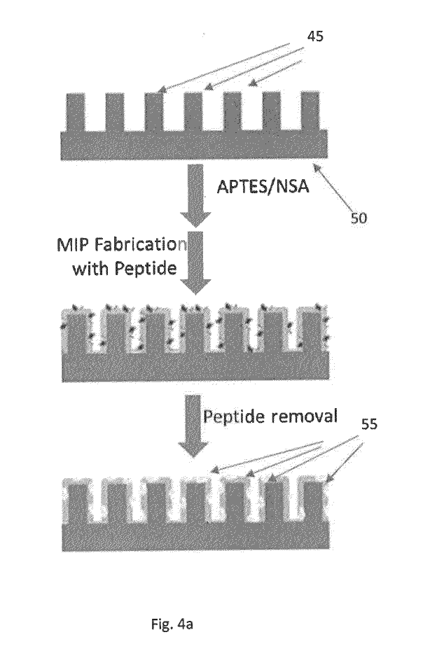

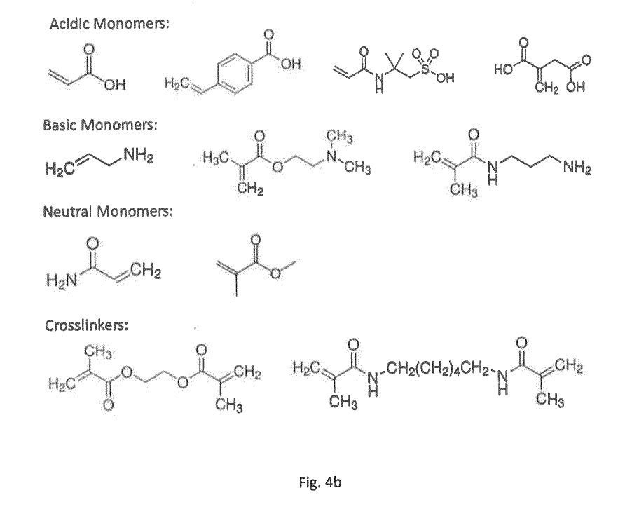

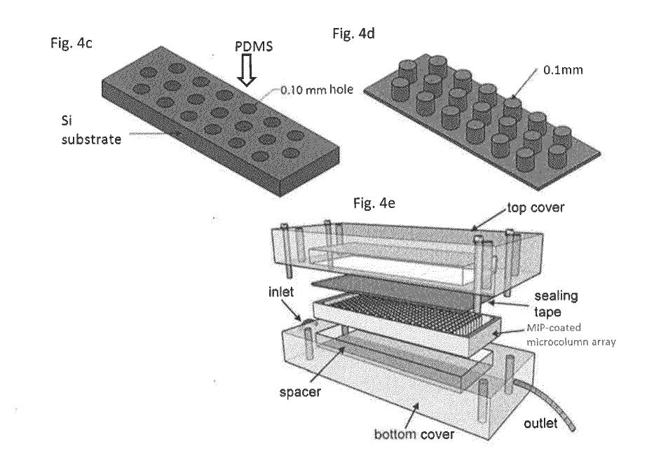

Molecularly-imprinted polymer (MIP) particles for capturing the target molecules were developed. The relevant fabrication involves the use of “emulsion polymerization” approach to synthesize MIP nanoparticles (Zeng, Z., et al. “Synthetic polymer nanoparticles with antibody-like affinity for a hydrophilic peptide.” ACS Nano 2010, 4 (1), pp 199-204, which is incorporated by reference herein in its entirety and for all purposes). An important aspect of emulsion polymerization is that it involves an aqueous solution of monomers dispersed in droplets in an immiscible organic solvent (e.g. toluene and hexane). The droplets are stabilized by surfactants. If a hydrophilic peptide is to be used as an imprint molecule, the peptide will be restricted to the water domain and as a consequence, no accessible binding sites will be formed. Therefore, the position occupied by the peptides at the interface of the water and oil domains during polymerization is very important to create accessible binding sites. To overcome this challenge, the target molecule (also referred to as a peptide) was first modified with fatty acid chains by amide coupling. In this scenario, the modified peptides function as surfactant molecules, with the hydrophobic tail in the oil domain and the hydrophilic segment (peptide) at the surface of the aqueous domain which contains the monomers (see FIG. 4). After polymerization, the MIP nanoparticles were cleaned and dialyzed to extract the imprinted peptide. The resultant nanoparticles were characterized using UV-Visible spectroscopy.

The microcolumn array was modified with peptide-imprinted polymers using the target molecule as the template molecule. In one instance, fabrication was done at the physiological pH (7.4). FIG. 4a schematically illustrates the steps to be used for the fabrication of the microfluidic channel, in the embodiment shown in FIG. 4a, in order to further elucidate these teachings, the exemplary embodiment of oxytocin purification is shown. Referring to FIG. 4a, in the embodiment shown therein, a microfluidic channel is formed by an array of microcolumns 45 disposed on a base 50. A complex having a peptide molecule (template) and a functional monomer is disposed on the surface of the microcolumns 45. After removal of the peptide, a molecularly imprinted polymer layer 55 is left on the surface of the microcolumns 45.

One challenge in forming the molecularly-imprinted polymer (MIP) coating layer is the optimizing the charge distribution, hydrophobicity and cross-link density to yield the highest purification efficiency for the target peptide. The molded PDMS channel was modified to introduce surface-bound acrylamide groups that covalently link the MIP to the channel wall.

A channel that can efficiently and specifically capture the target molecule was developed. A microfluidic channel consisting of micro-column arrays (FIGS. 4c-4e) was designed and fabricated. The microfluidic channel is used to separate and purify the target molecule (peptide). Two factors to be considered in order to achieve high capture efficiency are: (1) optimization of flow velocity to maximize frequency of contact between peptide and the molecularly-imprinted microcolumn array, and, (2) optimization of shear forces to ensure that they are lower than those favoring peptide capture to the recognition sites. Microcolumn size, spacing and the distribution along the streamlines are the critical variables that determine flow velocity and shear stress. The design of this microfluidic channel is similar to that used, for different purposes, in reported work (Sunitha, N. et al. “Isolation of rare circulating tumor cells in cancer patients by microchip technology,” Nature Letters 2007; 450: 1235-1239, which is incorporated by reference herein in its entirety and for all purposes) that showed the viability of separation of tumor cells in peripheral blood by fine control of the lamellar flow conditions. The micro-holes were fabricated on silicon substrate and molded silicone micro-columns were formed using a replication molding process on an etched silicon substrate (FIGS. 4c and 4d). The molded column uses the triangular pattern of cylindrical columns (100 μm), which are afterwards functionalized with a peptide-imprinted polymer or peptide-imprinted polymer nanoparticles.

Various monomers (see FIG. 4b) can be selected for MIP fabrication. In instances where the peptide is substantially deprotonated under physiological conditions (pH 7.4), (as in, for example, the 9-amino acid version. of oxytocin), thereby processing negative charge, basic monomers are selected. The pH of the precursor solution will be carefully controlled at the physiological condition (pH 7.4) to maintain the conformational and charge state of the template peptide during polymerization. The charge state, hydrophobicity and pore size of polymer can be modified by using different monomers and controlling the ratio of monomer/crosslinker based on the characterization and the purification performance. Although exemplary embodiments are presented below, a variety of monomers and cross-linkers are presented in the literature (see, for example, Kryscio, D. R. et al. “Critical review and perspective of macromolecularly-imprinted polymers.” Acta Biomaterialia 2012; 8: 461-473, which is incorporated by reference herein in its entirety and for all purposes).

In one or more embodiments, the method of these teachings includes disposing molecularly imprinted polymer nanoparticles on a surface of a structure, receiving, at the surface, a target fluid having the target molecules, capturing the target molecules in the molecularly imprinted polymer nanoparticles, releasing, after capture, the target molecules from the molecularly imprinted polymer nanoparticles, the target molecules being released into a release solution, providing the release solution to a sensor surface having a layer of molecular imprinted polymer disposed on the sensor surface, the target molecules binding to the layer of molecularly imprinted polymer, and detecting impedance changes in the layer of molecularly imprinted polymer caused by the binding of the target molecules to the molecularly imprinted polymer, the target molecules being detected by the impedance changes.

In order to better elucidate these teachings, the exemplary embodiment of detection of oxytocin is disclosed herein below. It should be noted that these teachings are not limited only to the exemplary embodiment.

Utilization of molecular imprinting for distinguishing oxytocin variants. The circumstance that the conformations and charges of peptide can be tuned by experimental conditions such as pH and ion concentration forms the key principle of our proposed approach. Researchers have taken advantage of this property to control nanocrystal growth by tuning peptide conformation. The principle is also verified by the large difference in isoelectric point (pI) between the OXT-9 (pI, 6.96) and OXT-12 (pI, 8.62). By tailoring the properties of polymers (by varying charge distribution or hydrophobicity or hydrophilicity or pore size), the formed specific binding site in molecularly imprinted polymer (MIP) matrix can record the conformation and charge state of the target peptide (FIG. 1a). This in turn enables distinguishing between the OXT-9 and OXT-12 versions. Equally importantly, the molecularly imprinted polymer in the purification stage is specifically tailored for the conformation and charge state of the target peptide in physiological conditions while the polymer for the detection is designed to be specific to the target molecule in the releasing solution. This unique combination further ensures the high specificity.

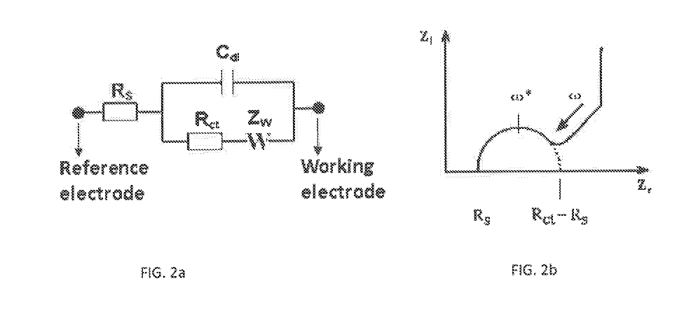

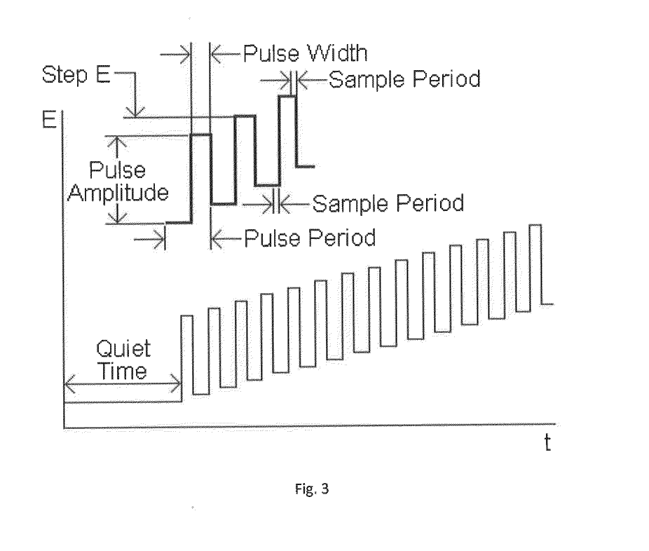

Ensuring the necessary sensitivity for oxytocin detection. Oxytocin levels in human body are in the pg/mL range, which requires a sensitive measurement method. In this work, sensitive detection of oxytocin was proposed to combine surface imprinting with electrochemical measurement. Imprinting a matrix with binding sites situated at the surface has been proven to have several unique advantages (e.g. easily accessible binding sites, rapid mass transfer and binding kinetics). Meanwhile, methods of electrochemistry impedance spectroscopy (EIS) and differential pulse voltammetry (DPV) were considered owing to their sensitive reliable properties and easy to miniaturization. The technical details about these two technologies are shown in FIGS. 2 and 3: EIS involves the application of an alternating voltage and monitoring of current response. The impedance response of systems is described using the ‘Randles equivalent circuit’ shown in FIG. 2, where Rₛ is the resistance of the electrolyte between the reference and the working electrode, C_{dl} is the double layer capacitance, and, R_{ct} is heterogeneous charge transfer resistance. Binding of the target molecule will result in change in one of these equivalent circuit parameters, As illustrated in FIG. 3, differential pulse voltammetry (DPV) consists of a series of regular voltage pulses superimposed on a staircase wave form. The current is measured immediately before each potential change, which difference is plotted as a function of potential.

A passive electrical system comprises both resistor and capacitor elements. Given the non-conductive nature of most biomolecules, the increase in the resistance occurs with increasing surface loading. Before oxytocin binding, the resistance is low because of the existence of highly conductive pathways from the solution to the gold conductive substrate. Once the targeted molecules bind to the cavities and block the conductive pathways, the resistance increases. Based the above hypothesis, both methods are suitable for detection of oxytocin.

Capture Stage

The molecularly imprinted polymer (MIP) particles were modified for capturing oxytocin. The relevant fabrication involves the use of “emulsion polymerization” approach to synthesize MIP nanoparticles. An important aspect of emulsion polymerization is that it involves an aqueous solution of monomers dispersed in droplets in an immiscible organic solvent (e.g. toluene and hexane). The droplets are stabilized by surfactants. If a hydrophilic peptide is to be used as an imprint molecule, the peptide will be restricted to the water domain and as a consequence, no accessible binding sites will be formed. Therefore, the position occupied by the peptides at the interface of the water and oil domains during polymerization is very important to create accessible binding sites. To overcome this challenge, oxytocin was first modified with fatty acid chains by amide coupling. In this scenario, the modified peptides function as surfactant molecules, with the hydrophobic tail in the oil domain and the hydrophilic segment (peptide) at the surface of the aqueous domain which contains the monomers (see FIG. 4). After polymerization, the MIP nanoparticles were cleaned and dialyzed to extract the imprinted peptide. The resultant nanoparticles were characterized using UV-Visible spectroscopy.

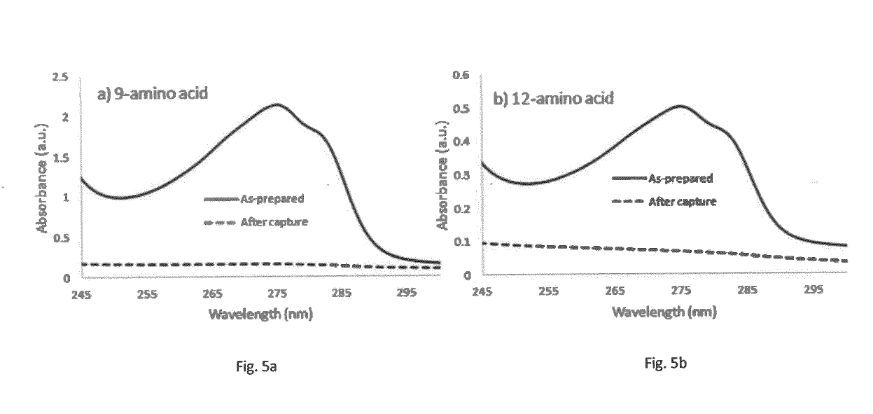

In order to determine the “Capture efficiency” of MIPs, UV-Visible spectroscopy studies were conducted. MIP particles were immobilized within a syringe filter. 100 μL of oxytocin solution (containing either 0.5 mg/ml of OXT-9 or OXT-12) was then carefully injected through immobilized MIP particles, followed by a thorough rinsing with Phosphate Buffered Saline (PBS) buffer (900 μL). The eluting PBS buffer was combined with the post-capture oxytocin solution for UV measurement (FIG. 5, dashed curve). For comparison, a control experiment was carried out, in which the volume (100 μL) of oxytocin solution (0.5 mg/ml) was directly diluted into 1 mL and tested by UV-Visible spectroscopy (FIG. 5, solid curve). Based on the absorbance at 275 nm of the UV-Visible spectra, it was calculated that the capturing efficiency was 92.7% for OXT-9 and 90.1% for OXT-12.

Release Efficiency for both OXT-9 and OXT-12 Forms after Capture

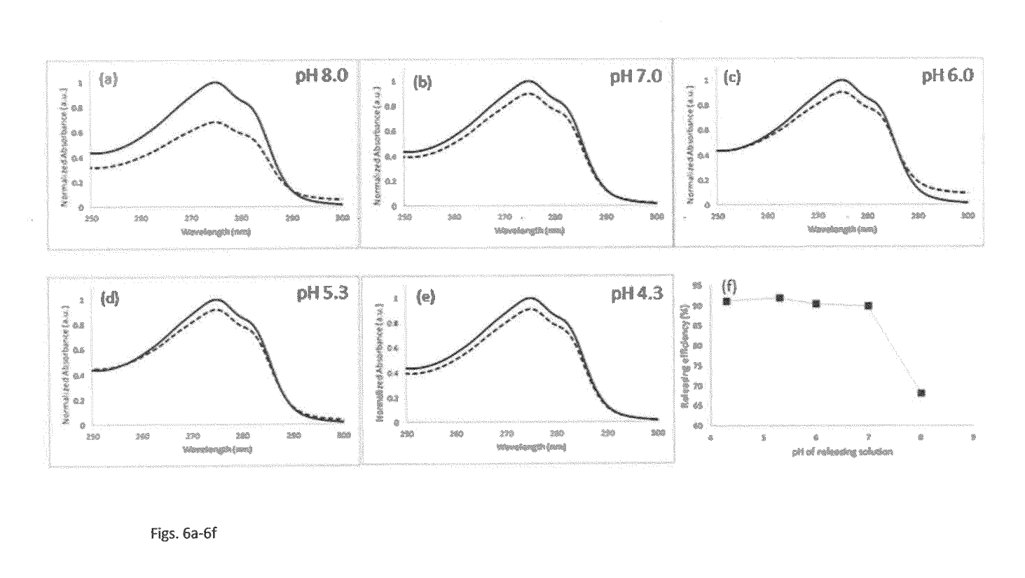

In the “capture and detection” scenario, the captured oxytocin shall be efficiently released and delivered for the detection. Therefore, an effective procedure to release most of captured oxytocin is critical. To do so, five capturing columns were prepared via immobilizing certain amount of cleaned and dialyzed MIP particles within syringe filters for each form of the oxytocin peptide. Meanwhile, five PBS solutions with different pH values were made for releasing the captured peptide. The typical procedure is described as followed: First, 100 μL of oxytocin solution (containing either 0.5 mg/ml of OXT-9 or OXT-12) was injected through a column, and then was thoroughly rinsed with PBS buffer (pH of 7.4) to remove any physically attached peptide. A releasing solution (1 mL) was then carefully injected through the above column and collected for UV characterization (dashed curves in FIGS. 6a-e and 7a-e). For comparison, a control experiment was carried out by directly diluting 100 μL of oxytocin solution into 1 mL with the corresponding as-prepared releasing solution and tested by UV-Visible spectroscopy (solid curves in FIGS. 6a-e and 7a-e). The releasing efficiency was therefore calculated based on the absorbance at 275 nm of these UV spectra, which correspond to certain pH value. The optimal pH for releasing 9-amino acid version is determined to be 5.3, which results in the efficiency of 91.9% and the most efficient release for 12-amino acid version is observed at pH of 8.9 with a value at 90.0%.

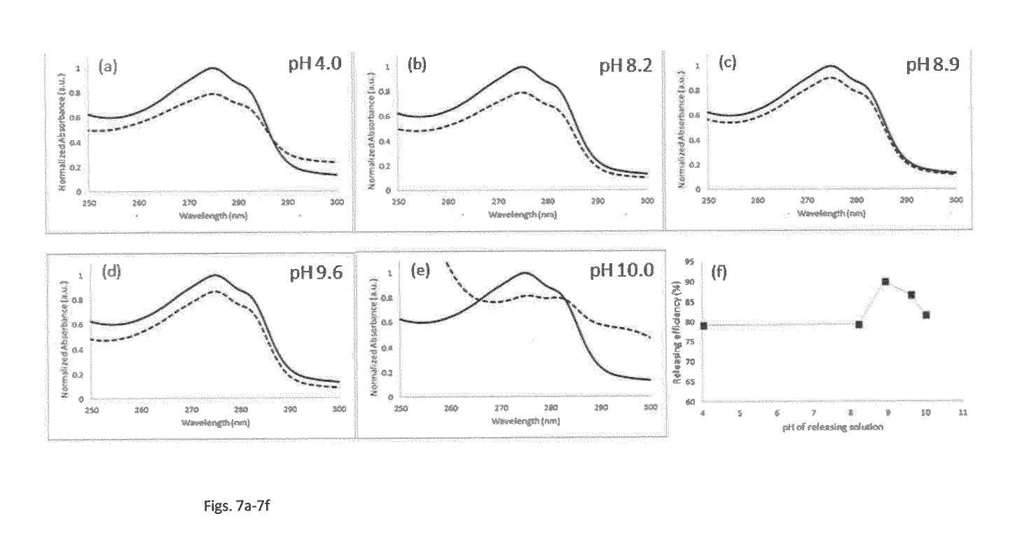

FIGS. 6f and 7f show the relationship between releasing efficiencies and pH values for OXT-9 and OXT-12, respectively, Such different releasing behaviors upon pH values are believed to link with the isoelectric point (pI) of oxytocin. Variance in isoelectric point induces difference in charge state and conformation of biomolecules (e.g. peptide and protein). The isoelectric point (pI) of peptide can be calculated based on the amino acid sequence. In the case of oxytocin, the calculated pI for OXT-9 is 6.96 while pI of OXT-12 is 8.62. Herein, under physiological condition (pH 7.4), OXT-9 is deprotonated while OXT-12 is protonated. Decreasing pH apparently protonates OXT-9 and induces drastic change in charge state and conformation of peptide, therefore leading to higher releasing efficiency for OXT-9. In contrast to OXT-9, OXT-12 is protonated in pH 7,4, which indicates that increasing pH to deprotonate captured analytes is the effective way to change their charge state and conformation for higher releasing efficiency.

Specificity of Capture

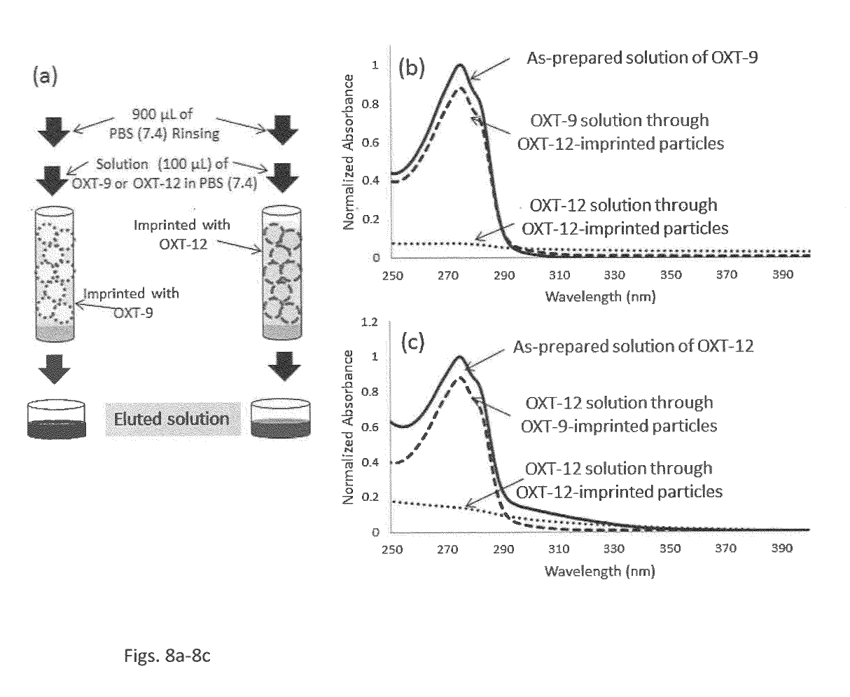

Current immunoassays fail to differentiate the neuroactive 9-amino acid version (OXT-9) from the pre-hormone 12-aminoacid version (OXT-12). The reason is due to the nature of antibody: the specific recognition ability of antibodies relies on a short variable sequence of amino acids at the tips of the Y-structure [6], which is called the paratope and specific for one particular moiety of the analyte. In the scenario of oxytocin, OXT-9 and OXT-12 both can bind to the paratope of antibody with a similar affinity because both consist of an identical amino acid tip segment. Consequently, immunoassay does not have the specificity to discriminate between the OXT-9 and OXT-12 forms, In other words, they do not have the specificity required by DARPA. To ascertain that the capture stages described in the previous sections do have the specificity needed, two separate columns were prepared with particles imprinted for OXT-9 and OXT-12 (FIG. 8). A test solution (100 μL), containing either OXT-9 or OXT-12 (0.5 mg/ml) in PBS (pH 7.4) buffer, was carefully injected through the columns, followed by the PBS buffer (pH 7.4, 900 μL) rinsing. The eluting solutions through column were collected and characterized separately using UV spectroscopy. For comparison, 100 μL of oxytocin solution (0.5 mg/ml OXT-9 or OXT-12 in PBS buffer) was directly diluted into 1 mL using PBS (7.4) and measured by UV-Visible spectroscopy (solid lines in FIG. 8b and c, indicated with as-prepared). It can be clearly seen that while there is no absorption peak for the OXT-9 solution injected through the OXT-9 imprinted column, there is hardly a change in the absorption strength for the OXT-9 solution through the OXT-12 imprinted column. Similar results were obtained from the experiments using the OXT-12 solution—there is no absorption peak observed for the OXT-12 after injecting through the OXT-12 imprinted column while there is hardly a change in the absorbance for the OXT-12 solution through the OXT-9 imprinted column.

Specificity from Mass Spectrometry Studies

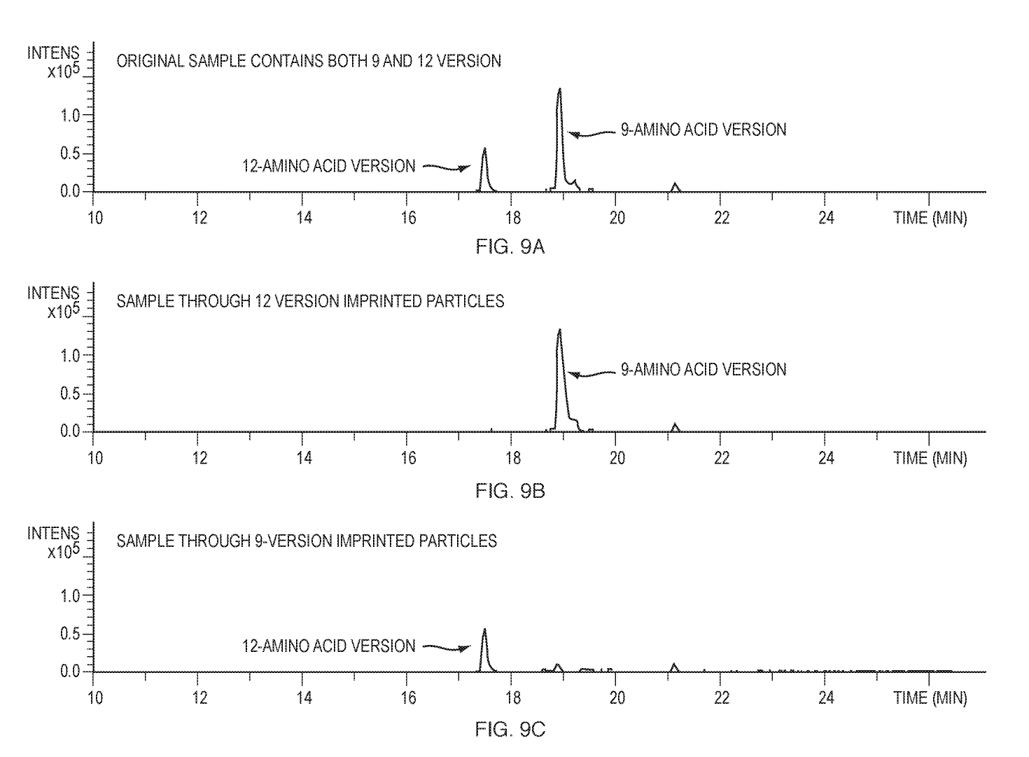

While the previous section showed that the capture specificity for both OXT-9 and OXT-12 forms, these were based on the UV absorption studies. The capture process was optimized and the specificity determined by a more accurate method—mass spectrometry. For this purpose, a sample solution containing both OXT-9 (0.5 mg/mL) and OXT-12 versions (0.5 mg/mL) in PBS (pH 7.4) was prepared along with two capturing columns (one with OXT-9 imprinted particles and the other with OXT-12 imprinted). 100 μL of test oxytocin sample solution was then carefully injected through a capture column, followed by the thorough rinsing with use of 0.9 ml of PBS buffer (pH 7.4). The rinsing PBS buffer was collected and combined with the post-capture sample solution for mass spectroscopy measurement. For comparison, 100 μL of oxytocin sample solution was directly diluted into 1 mL and characterized with mass spectroscopy (FIG. 9a).

The ultra-high pressure liquid chromatography coupled with time of flight high-resolution mass spectroscopy (UPLC-QtoF HRMS) was applied to characterize the above solutions. The stationary phase was a C-18 column and the mobile phase was a gradient of water and acetonitrile. Mass spectroscopic studies clearly show that OXT-12-imprinted particles preferentially capture OXT-12 version while OXT-9-imprinted particles preferentially capture OXT-9 version

Sensitive Detectors

To ensure the necessary sensitivity, the critical challenge here was to form an ultra-thin and uniform molecularly-imprinted polymer (MIP), which is capable of sensitively transducing binding events into a detectable electronic signal. The ideal polymer material for coating on the sensor electrode should have the following properties: (1) It should be insulating and (2) It should form a thin and uniform layer. Polyphenol (PPn) yrs MIP for Sensitive detectors was used.

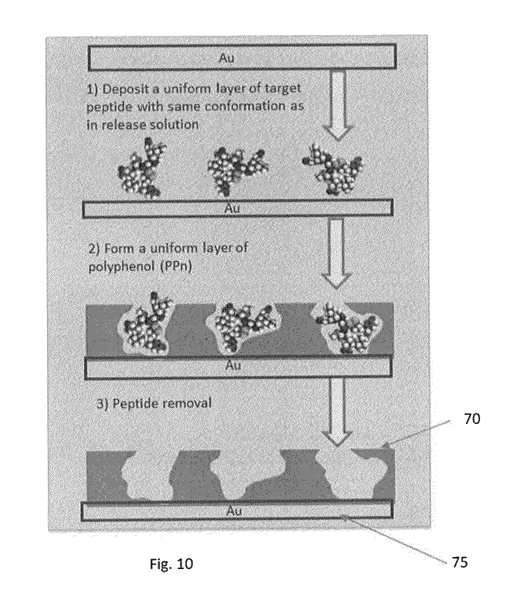

The electrochemical coating of PPn on the flat gold surface is expected to be highly uniform and ultra-thin, due to the self-limiting nature of the deposition. This technical approach has been experimentally demonstrated on a more challenging surface (CNT arrayed architecture) (see, for example, Dong, C. et al, “A molecular-imprint nanosensor for ultrasensitive detection of proteins.” NATURE NANOTECHNOLOGY 2010; 5: 597-601, which is incorporated by reference herein in its entirety and for all purposes). A typical procedure is schematically shown in FIG. 10. In the embodiment shown in FIG. 10, in order to further elucidate these teachings, the exemplary embodiment of oxytocin detector is shown.

The oxytocin detectors were constructed via electrochemically depositing a layer of oxytocin-imprinted polyphenol (PPn) on a flat gold surface. The experimentally demonstrated technical approach (Dong, C. et al. “A molecular-imprint nanosensor for ultrasensitive detection of proteins.” NATURE NANOTECHNOLOGY 2010; 5: 597-601) was applied, which is briefly described below: In a three-electrode electrochemical system, oxytocin peptide was first attracted onto gold surface. Cyclic voltammetry was then applied in presence of phenol monomer at a scanning rate of +30 mV s⁻¹ between 0.0 to 0.9 V versus the reference electrode (Ag wire). The deposited PPn layer has shown to be highly uniform and ultra-thin, due to the self-limiting nature of the electrochemical polymerization. The resultant detector was rinsed and incubated overnight in deionized water to remove the imprinted peptides. The resultant detector has a layer of molecular imprinted polymer 70 disposed on a conductive surface 75, a flat gold surface in the embodiment shown.

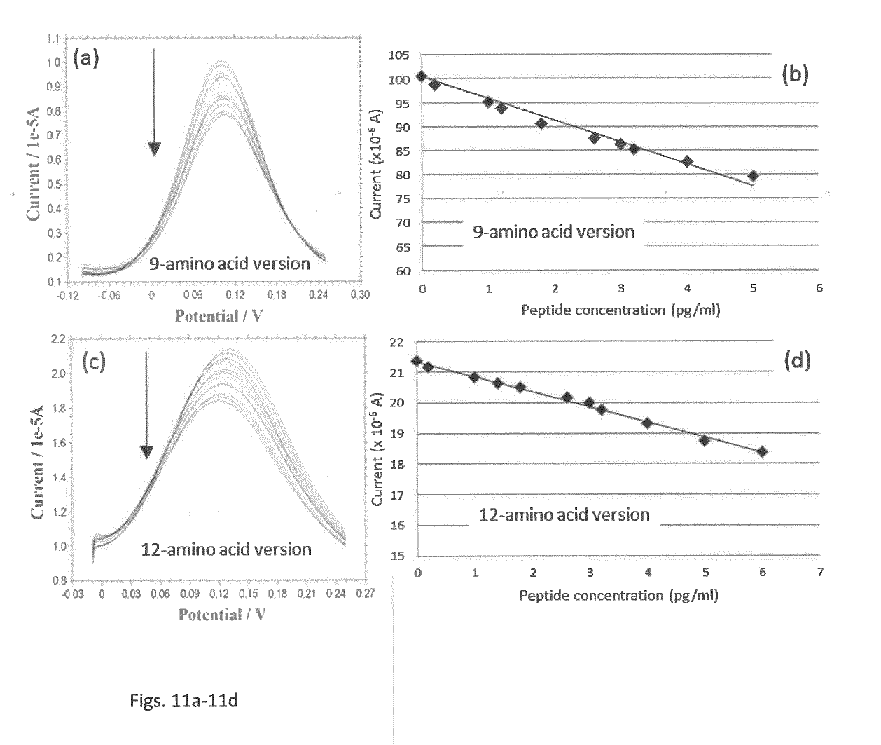

Sensitivity: The detection of oxytocin binding to its imprint site on the detector surface was evaluated using differential pulse voltammetry (DPV). A three-electrode electrochemical system was configured by connecting the sensor (gold substrate with polyphenol (PPn) coating) as the working electrode, using silver (Ag) as the reference electrode and platinum (Pt) wire as the counter electrode. FIG. 11 shows the DPV data on the detection for both OXT-9 and OXT-12. The experiment was conducted by successively adding oxytocin of known concentrations. Each such addition led to a decrease in current. The sensitivity for the detection of both OXT-9 and OXT-12 versions was demonstrated by two independent series of measurements (FIGS. 11a and c). In each case, the target version of oxytocin was detected to a low concentration of 0.2 pg/ml as illustrated in the plots of peak current with concentrations (FIGS. 11b and d). The change of permittivity and resistivity in the surface materials in response to oxytocin binding is considered as the primary mechanism of signaling (oxytocin molecules have lower permittivity and higher resistivity than the replaced water in the imprint sites, leading to decreased capacitance and increased resistance).

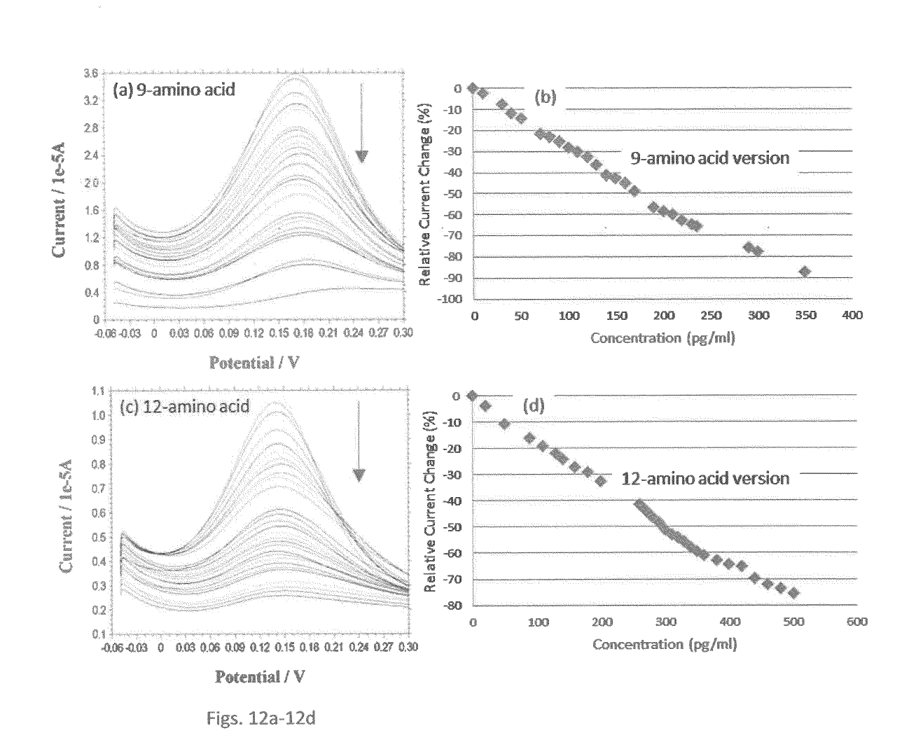

Dynamic range: Based on a literature survey, oxytocin levels in plasma or saliva appear to range from a few pg/mL several to approximately three hundred pg/ml. Such a broad variation indicates the importance of the sensing dynamic range for practical applications. Similarly, differential pulse voltammetry (DPV) was used to determine the sensing dynamic range within a three-electrode configuration. FIG. 12 shows the studies on the dynamic range of detectors imprinted with OXT-9 and OXT-12 versions, respectively. Successively adding oxytocin in concentrations revealed a concentration-dependent decrease in current. The dynamic range for the detection of both OXT-9 and OXT-12 was demonstrated in two independent series of measurements (FIGS. 12a and c). In each case, presence of target version of oxytocin up to hundreds pg/ml can be-detected. The observed phenomenon is in close agreement with the previous research work on the determination of specific protein using DPV method (Dong, C. et al. “A molecular-imprint nanosensor for ultrasensitive detection of proteins,” NATURE NANOTECHNOLOGY 2010: 5: 597-601).

The relative peak current changes were calculated and plotted with the oxytocin levels.

FIGS. 12b and d show the dependence of the calculated relative current change on the oxytocin concentration in their corresponding PBS buffers. As given in DPV measurements, both detection of OXT-9 and OXT-12 version can be as high as hundreds pg/ml in PBS buffer. Thus, with the demonstrated sensitivity of 0.2 pg/ml and large dynamic range, the sensor has great potential for sensitively quantifying oxytocin levels in plasma or saliva.

Integration Design of Capture and Detection Stages into a Common Platform.

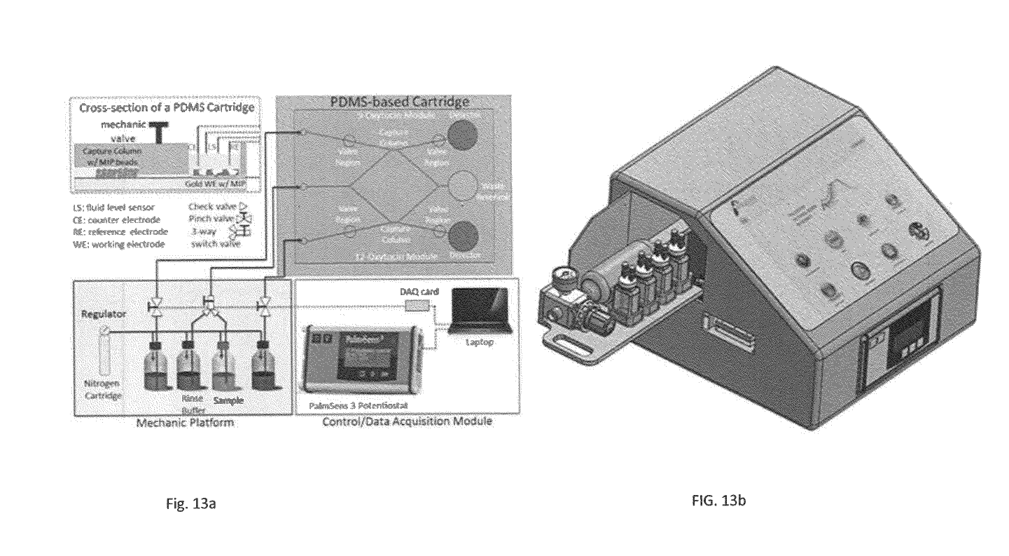

The integrated device will include three major modules—a mechanical platform for fluid handling, and a cartridge containing capture column and electrochemical detector, and a data acquisition module containing an electrochemical workstation, a laptop, and a data acquisition card (FIG. 13a).

The integration approach, in one instance, involves several steps: i) Conversion of the first version of the cartridge to a PDMS-based microfluidic cartridge, which will render the process amenable for manufacturing, ii) Automatic control of the flow of fluids in the mechanical platform, and iii) development of the hardware and the software necessary to enable a fully automatic data collection.

Although these teachings have been described with respect to various embodiments, it should be realized these teachings are also capable of a wide variety of further and other embodiments within the spirit and scope of the appended claims.

What is claimed is:

1. A sensor for detecting and recognizing target molecules, the sensor comprising:
a capture and release components comprising:
a structure having one of molecularly imprinted polymer layer or molecularly imprinted polymer nanoparticles disposed on the structure; the structure being configured to receive a target fluid having the target molecules; the target molecules being captured by the molecularly imprinted polymer nanoparticles;
the structure being also configured to receive a release solvent, the release solvent releasing the target molecules captured by the molecularly imprinted polymer nanoparticles; the release solvent and released target molecules constituting a release solution; and
a sensing component comprising:
a sensor surface having a layer of molecular imprinted polymer disposed on a sensor surface; the layer of molecularly imprinted polymer disposed to receive the release solution; the target molecules binding to the molecularly imprinted polymer; and
a sensing circuit configured to detect impedance changes in the layer of molecularly imprinted polymer caused by t binding of the target molecules to the molecularly imprinted polymer;
the capture and release components operatively connected to receive from a fluid source the target fluid or the release solvent; the sensing component operatively connected to the capture and release components in order to receive the release solution.

2. The sensor of claim 1 wherein the structure comprises a micro fluidic channel comprising an array of micro columns disposed on a base.

3. The sensor of claim 2 wherein micro column size, spacing between micro columns and distribution of micro columns along streamlines are selected to increase frequency of contact between the target molecules and molecularly imprinted material and to resolve in shear forces that favor target molecule capture and recognition sites in the molecularly imprinted material, the molecularly imprinted material being one of the molecularly imprinted polymer layer or in the molecularly imprinted polymer nanoparticles disposed on a surface of the micro columns.

4. The sensor of claim 3 wherein the micro columns are modified by surface bound acrylamide groups configured to covalently link the molecularly imprinted material to the surface of the micro columns.

5. The sensor of claim 4 wherein the molecularly imprinted material comprises molecularly imprinted polymer nanoparticles.

6. The sensor of claim 4 wherein the target molecules comprise oxytocin.

7. The sensor of claim 1 wherein the sensor surface is a surface of a conductive material.

8. The sensor of claim 7 wherein the conductive material is gold.

9. The sensor of claim 8 wherein the layer of molecularly imprinted polymer is comprises a layer of polyphenol (PPn).

10. A method for detecting and recognizing target molecules, the method comprising;
receiving, at a surface of a structure, a target fluid having the target molecules; the structure having one of molecularly imprinted polymer layer or molecularly imprinted polymer nanoparticles disposed on the surface;
capturing the target molecules in the molecularly imprinted polymer nanoparticles;
releasing, after capture, the target molecules from the molecularly imprinted polymer nanoparticles, the target molecules being released into a release solution;
providing the release solution to a sensor surface having a layer of molecular imprinted polymer disposed on the sensor surface; the target molecules binding to the layer of molecularly imprinted polymer; and
detecting impedance changes in the layer of molecularly imprinted polymer caused by the binding of the target molecules to the molecularly imprinted polymer; the target molecules being detected by the impedance changes.

11. The method of claim 10 wherein the structure comprises a micro fluidic channel comprising an array of micro columns disposed on a base.

12. The method of claim 11 wherein micro column size, spacing between micro columns and distribution of micro columns along streamlines selected to increase frequency of contact between the target molecules and molecularly imprinted material and to resolve in shear forces that favor target molecule capture and recognition sites in the molecularly imprinted material, the molecularly imprinted material being one of the molecularly imprinted polymer layer or in the molecularly imprinted polymer nanoparticles disposed on a surface of the micro columns.

13. The method of claim 12 wherein the micro columns are modified by surface bound acrylamide groups configured to covalently link the molecularly imprinted material to the surface of the micro columns.

14. The method of claim 13 wherein molecularly imprinted material comprises molecularly imprinted polymer nanoparticles.
